(19) United States
(12) Patent Application Publication
(10) Pub. No.: US 2026/0009156 A1
(43) Pub. Date: Jan. 8, 2026

(54) HYDROGEN SENSOR MATERIAL

(71) Applicant: Christian-Albrechts-Universitaet Zu Kiel, Kiel (DE)

(72) Inventors: Mirjam Patricia Margarete POSCHMANN, Kiel (DE); Leonard SIEBERT, Altenholz (DE); Cristian LUPAN, Chisinau (MD); Oleg LUPAN, Chisinau (MD); Fabian SCHUETT, Kiel (DE); Rainer ADELUNG, Kiel (DE); Norbert STOCK, Kiel (DE)

(73) Assignee: Christian-Albrechts-Universitaet Zu Kiel, Kiel (DE)

(21) Appl. No.: 19/328,607

(22) Filed: Sep. 15, 2025

Related U.S. Application Data
(63) Continuation of application No. PCT/DE2024/100199, filed on Mar. 13, 2024.

(30) Foreign Application Priority Data
Mar. 14, 2023 (DE) 10 2023 106 401.0

(51) Int. Cl. C30B 29/16; C07F 3/06; C09D 7/63; C30B 29/60; C30B 33/00; G01N 33/00

(52) U.S. Cl. CPC C30B 29/16; C07F 3/06; C09D 7/63; C30B 29/60; C30B 33/00; G01N 33/005

(57) ABSTRACT

A hydrogen sensor material comprising at least one rod-shaped single crystal with a wurtzite structure made of a semiconductor and a conversion layer formed by a MOF on the surface of the single crystal. The single crystal has a length of 100 nm to 100 μm and a lateral extent of 500 nm to 10 μm. The aspect ratio of the single crystal of length to lateral extent is greater than 3. Furthermore, the invention relates to a production method for a hydrogen sensor material.

This nonprovisional application is a continuation of International Application No. PCT/DE2024/100199, which was filed on Mar. 13, 2024, and which claims priority to German Patent Application No. 10 2023 106 401.0, which was filed in Germany on Mar. 14, 2023, and which are both herein incorporated by reference.

### BACKGROUND OF THE INVENTION

### Field of the Invention

The invention relates to a new sensor material for a hydrogen sensor.

### Description of the Background Art

In the course of the transformation of the energy sector from fossil to renewable, carbon-free energy sources, hydrogen (H₂) is becoming increasingly important. For example, it is planned to use the existing infrastructure and save up to 20% H₂ to feed into natural gas pipelines. In this context, the precise detection of even the smallest amounts of H₂ is of great importance, among other things because leaks in the pipes must be detected immediately.

Gas sensors based on zinc oxide nanostructures are seen as promising candidates. See V. S. Bhati, M. Hojamberdiev, M. Kumar, Enhanced sensing performance of ZnO nanostructures-based gas sensors: A review, Energy Rep. 6 (2020) 46-62.

A disadvantage of gas sensors made of semi-conducting metal oxides (MOS) is that they have low selectivity towards reducing gases, i.e., these are detected but cannot be distinguished from one another. One approach to solving this problem is molecular filters made of metal-organic frameworks (MOFs), which are located as a conversion layer at the gas-sensor interface to prevent contamination of the signal.

US 2021/0016245 describes MOFs coated on various substrates and the use of these coated substrates as sensor material.

CN107991350A discloses rod-shaped sensor material made of zinc oxide (ZnO) coated on the surface with the metal-organic framework compound ZIF-8 ([Zn(C₄H₅N₂)₂)]) (ZnO@ZIF-8).

Further zinc oxide structures coated with ZIF-8 (ZnO@ZIF-8) intended as sensor material for the detection of hydrogen are known from the following publications.

F. Cui, W. Chen, L. Jin, H. Zhang, Z. Jiang, Z. Song, Fabrication of ZIF-8 encapsulated ZnO microrods with enhanced sensing properties for H₂ detection, J. Mater. Sci. Mater. Electron. 29 (2018) 19697-19709. https://doi.org/10.1007/s10854-018-0095-9 discloses that zinc oxide is a popular sensor material with limited sensitivity, selectivity, and stability for gas detection, especially H2 detection, while zeolite imidazolate framework-8 (ZIF-8), a type of metal-organic frameworks (MOFs), possesses tunable porosity, large specific surface area, and good thermal stability.

FREUND, Ralph et al.: Understanding the Chemistry of Metal Oxide to Metal-Organic Framework Reactions for Morphology Control. In: Chemistry of Materials, 2023, 35, 1891-1900. DOI: 10.1021/acs.chemmater.2c02946 discloses that metal-organic frameworks (MOFs) are a class of porous materials of which the three-dimensional (3D) morphological control could be of interest. Complex 3D ZIF-8 and ZnO@ZIF-8 composite structures composed of different ZnO precursors are conceivable.

M. Drobek, J.-H. Kim, M. Bechelany, C. Vallicari, A. Julbe, S. S. Kim, MOF-Based Membrane Encapsulated ZnO Nanowires for Enhanced Gas Sensor Selectivity, ACS Appl. Mater. Interfaces. 8 (2016) 8323-8328. https://doi.org/10.1021/acsami.5b12062

P. Ji, X. Hu, R. Tian, H. Zheng, J. Sun, W. Zhang, J. Peng, Atom-economical synthesis of ZnO@ZIF-8 core-shell heterostructure by dry gel conversion (DGC) method for enhanced H₂ sensing selectivity, J. Mater. Chem. C. 8 (2020) 2927-2936. https://doi.org/10.1039/C9TC06530J

X. Wu, S. Xiong, Z. Mao, S. Hu, X. Long, A Designed ZnO@ZIF-8 Core-Shell Nanorod Film as a Gas Sensor with Excellent Selectivity for H₂ over CO, Chem.—Eur. J. 23 (2017) 7969-7975. https://doi.org/10.1002/chem.201700320

R. Lv, Q. Zhang, W. Wang, Y. Lin, S. Zhang, ZnO@ZIF-8 Core-Shell Structure Gas Sensors with Excellent Selectivity to H₂, Sensors. 21 (2021) 4069. https://doi.org/10.3390/s21124069

A. I. Khudiar, A. K. Elttayef, M. K. Khalaf, A. M. Oufi, Fabrication of ZnO@ZIF-8 gas sensors for selective gas detection, Mater. Res. Express. 6 (2020) 126450. https://doi.org/10.1088/2053-1591/ab69c2

MISHRA, Yogendra Kumar [et al.]: Direct Growth of Freestanding ZnO Tetrapod Networks for Multifunctional Applications in Photocatalysis, UV Photodetection, and Gas Sensing. In: ACS Applied Materials and Interfaces 2015, 7, 14303-14316: DOI: 10.1021/acsami.5b02816.

RASCH, Florian [et al.]: Highly selective and ultra-low power consumption metal oxide based hydrogen gas sensor employing graphene oxide as molecular sieve. In: Sensors & Actuators: B. Chemical, 2020, 320, 128363. DOI: 10.1016/j.snb.2020.128363.

MENG, Jiashen [et al.]: Advances in metal-organic framework coatings: versatile synthesis and broad applications. In: Chemical Society Review, 2020, 49, 3142. DOI: 10.1039/c9cs00806c.

A disadvantage of the known sensor materials for hydrogen sensors is the working temperature of the sensor, which in some cases is even well above 120° C. Especially at high concentrations of H₂, high sensor temperatures pose a significant risk due to flammability. Another disadvantage is that heating to high temperatures consumes more energy. A further disadvantage of the known sensor materials for hydrogen sensors is the high cross-sensitivity to other gases and thus low selectivity. Furthermore, it is disadvantageous that all known sensor materials for hydrogen sensors require oxygen to be present during detection. In particular, the sensitivity of known sensor materials for hydrogen sensors needs to be improved.

### SUMMARY OF THE INVENTION

It is therefore an object of the invention to provide a hydrogen sensor material which has an increased sensitivity compared to all known hydrogen sensor materials.

Furthermore, it is an object of the invention to provide a hydrogen sensor material that provides a high selectivity of the sensor.

Furthermore, it is an object of the invention to provide a hydrogen sensor material that can be used in a working range of less than 120° C.

Another object of the invention is to provide a hydrogen sensor material that can also be used in the absence of oxygen.

The object of the invention is achieved, in an example, by a hydrogen sensor material comprising at least one rod-shaped single crystal with a wurtzite structure made of a semiconductor and a conversion layer formed by a MOF on the surface of the single crystal, wherein the single crystal has a length of 100 nm to 100 μm and a lateral extent of 500 nm to 10 μm, and wherein the aspect ratio of the single crystal of length to lateral extent is greater than 3.

In an example, the object of the invention is achieved by a hydrogen sensor material comprising at least one rod-shaped single crystal with wurtzite structure selected from the group ZnO, ZnTe, ZnSe.

In an example, the object of the invention is achieved by a hydrogen sensor material comprising at least one rod-shaped single crystal with a wurtzite structure made of ZnO.

In an example, the object of the invention is achieved by a hydrogen sensor material comprising at least one rod-shaped single crystal with a wurtzite structure made of zinc oxide (ZnO) and a conversion layer, formed by the MOF ZIF-8 ([Zn(C₄H₅N₂)₂)]), on the surface of the single crystal.

In an example, the object of the invention is achieved by a hydrogen sensor material comprising at least one rod-shaped single crystal with a wurtzite structure from the arm or parts thereof of a tetrapodal zinc oxide (t-ZnO) and a conversion layer, formed by the MOF ZIF-8 ([Zn(C₄H₅N₂)₂)]), on the surface of the single crystal, wherein the single crystal has a length of 100 nm to 100 μm and a lateral extent of 500 nm to 10 μm, and wherein the aspect ratio of the single crystal of length to lateral extent is greater than 3.

In an example of the invention, several single crystals according to the invention are connected to form a diffusion-open network.

A person skilled in the art understands semiconductors to be solid-state materials which have a medium band gap greater than 0 eV and which behave like an electrical insulator at a temperature of 0 K and like an electrical conductor at a finite temperature below the melting point of the material, preferably not more than 30° C. from the application temperature. The average conductivity at this temperature is usually between that of an insulator and an electrical conductor.

The wurtzite structure is understood by a person skilled in the art to be the structure of compounds of the composition AB, which are in the space group P6₃mc or space group number 186, i.e., crystallize in a hexagonal crystal system. Each type of atom is surrounded tetrahedrally by the other type of atom.

The electrical properties of semiconductor single crystals can be characterized by the degree of their defect density. The defect density can be determined via the decay time of the photoluminescence. See [Zhong et al. J. Phys. Chem. C. 2008, 112, 16286-16295], which is incorporated herein by reference. The rod-shaped single crystals with wurtzite structure according to the invention have a decay constant of more than 1 nm, determined by photoluminescence.

The sensitivity of the sensor can be understood to be the extent to which the electrical resistance is switched by the sensor with and without the presence of the analyte at a fixed concentration. The measure of sensitivity is the quotient of the conductivities and corresponds to the sensor response.

The selectivity of the sensor refers to the comparison of the measured sensitivities for different gases. If a sensitivity of 1 is shown for a gas, this means that the resistance does not change after adding this gas, so the sensor shows no sensitivity to this gas, the cross sensitivity is minimal in this case and the selectivity is maximum.

Surprisingly, it was found that the hydrogen sensor material according to the invention has a sensitivity increased by a factor of 50 to 100 compared to known sensor materials for hydrogen sensors.

Also surprisingly, it was found that the hydrogen sensor material according to the invention has excellent selectivity compared to known sensor materials for hydrogen sensors. The sensitivities for the gases methane, acetone, ethanol, 2-propanol, n-butanol, ammonia and CO₂ are 1. This shows an almost infinite selectivity with respect to the gas hydrogen.

Surprisingly, it was found that the hydrogen sensor material according to the invention can be used for hydrogen sensors at operating temperatures of less than 120° C., compared to known sensor materials.

It was also surprisingly found that the hydrogen sensor material according to the invention can be used for measurement even in the absence of oxygen, compared to known sensor materials for hydrogen sensors.

In a further aspect, the object of the invention is achieved by a method for producing a hydrogen sensor material comprising the following steps.
I. Determination of the reaction temperature in the range between 60° C. and 160° C., the reaction time and the molar ratio in relation to the desired layer thickness based on Table 1, which shows the minimum ratio of ZnO to HMeIM, an increase in the proportion of HMeIM is possible without changing the overall result and Table 2 and the RGT rule:

TABLE 1
ZnO to HMelM ratio

| reaction time/h | ZnO | HMelM |
|---|---|---|
| 4 | 50 | 1 |
| 5 | 40 | 1 |
| 10 | 25 | 1 |
| 15 | 25 | 2 |
| 20 | 10 | 1 |
| 30 | 6 | 1 |
| 40 | 5 | 1 |
| 50 | 10 | 3 |
| 60 | 2 | 1 |

TABLE 2
Average layer thickness of the ZIF-8 coating, which at a reaction
temperature of 140° C. can be achieved for a given reaction time.

| | Average layer thickness at a | |
|---|---|---|
| | reaction temperature of | |
| Reaction time/h | 140° C./nm | |
| 4 | 60 | (±20) |
| 8 | 160 | (±80) |
| 10 | 210 | (±110) |
| 15 | 320 | (±160) |
| 20 | 430 | (±220) |

II. Placing ZnO material in a sealable reactor
III. Addition of 2-methylimidazole (HMeIM),
IV. Closing the reactor
V. Temperature control of the reactor
VI. Cooling the reactor
VII. Optionally: Removal of excess HMeIM by treating the crude product under reduced pressure and/or at elevated temperature,
wherein steps II and III can be carried out in a variable order.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows SEM images of the sensor material t-ZnO@ZIF-8;

FIG. 2 is a schematic representation of the method according to the invention for producing the sensor material (t-ZnO@ZIF-8);

FIG. 3 shows SEM images of a t-ZnO@ZIF-8(4h) microrod (a) brought into contact with platinum via the FIB with the gold contacts on a SiO2/silicon wafer (b) cross-section of a t-ZnO@ZIF-8(4h) microrod;

FIG. 4 shows a response behavior of the sensor according to the invention for different gases;

FIG. 5 shows a response times and regeneration times of the sensor according to the invention; and

FIG. 6 shows a response behavior of the sensor according to the invention in an oxygen-free atmosphere

### DETAILED DESCRIPTION

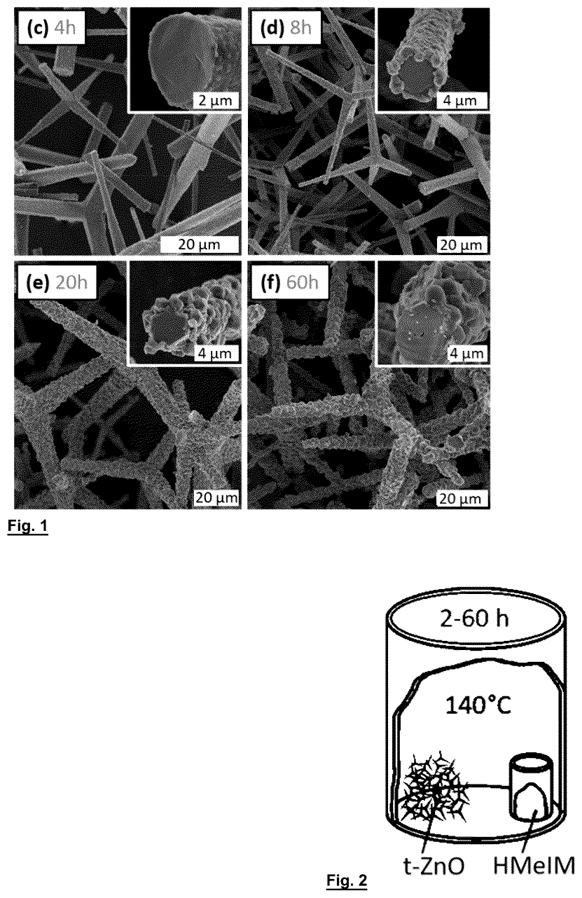

FIG. 1 shows the SEM images of the single crystals according to the invention, here the tetrapods of zinc oxide t-ZnO, which at the surface have a conversion layer formed by the MOF ZIF-8 ([Zn(C₄H₅N₂)₂)]). (t-ZnO@ZIF-8) is shown after 4 hours (c), 8 hours (d), 20 hours (e) and 60 hours reaction time (f).

The images show the good homogeneity of the ZIF-8 coating. The thickness of the layers can be found in the following table.

TABLE 3
Thickness of the ZIF-8 layer on the ZnO tetrapods

| Reaction time/h | Average layer thickness/nm | Standard deviation/nm | Median/nm |
|---|---|---|---|
| 4 | 57 | 24 | 49 |
| 8 | 175 | 89 | 148 |
| 20 | 398 | 204 | 375 |
| 60 | 1026 | 384 | 1011 |

The quantitative determination of the ZIF-8 content was carried out by thermogravimetric analysis. The samples contain the amounts of ZIF-8 listed in Table 2.

TABLE 4
Content of ZIF-8 in t-ZnO@ZIF-8 determined by TG analysis

| Reaction time/h | Δm/% | w(ZIF-8)/% |
|---|---|---|
| 4 | 1.2 | 1.8 |
| 5 | 1.8 | 2.7 |
| 10 | 2.5 | 3.9 |
| 15 | 6.0 | 9.4 |
| 20 | 7.2 | 11.2 |
| 30 | 9.9 | 15.4 |
| 40 | 12.1 | 18.9 |
| 50 | 21.0 | 32.6 |
| 60 | 24.2 | 37.7 |

The specific surface areas were determined by nitrogen sorption measurement and are listed in Table 5.

TABLE 5
Specific surface area A_{S, BET} of the t-ZnO@ZIF-8
samples as a function of reaction time

| t/h | A_{S, BET}/m²/g |
|---|---|
| 4 | 26 |
| 5 | 34 |
| 10 | 58 |
| 15 | 122 |
| 20 | 160 |
| 30 | 250 |
| 40 | 316 |
| 50 | 604 |
| 60 | 761 |

FIG. 2 shows a schematic representation of the method according to the invention for producing the sensor material also according to the invention (here t-ZnO@ZIF-8).

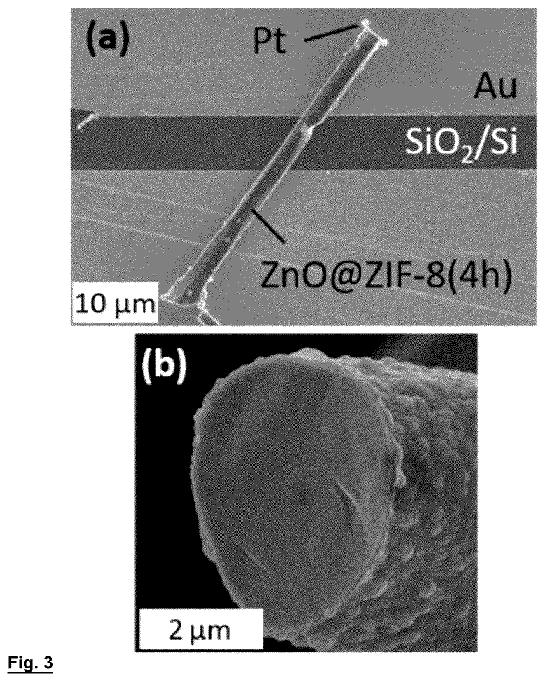

For the measurement of hydrogen detection of the sample t-ZnO@ZIF-8(4h), an arm of a single tetrapod was connected by means of a focused ion beam (FIB) method on a SiO₂/silicon wafer to gold contacts to measure the current flow in a single tetrapod when exposed to different sample gases. FIG. 3(a) shows the SEM images of a t-ZnO@ZIF-8(4h) microrod brought into contact with platinum via the FIB method with the gold contacts on a SiO₂/silicon wafer. FIG. 3(b) shows the cross-section of a t-ZnO@ZIF-8(4h) microrod.

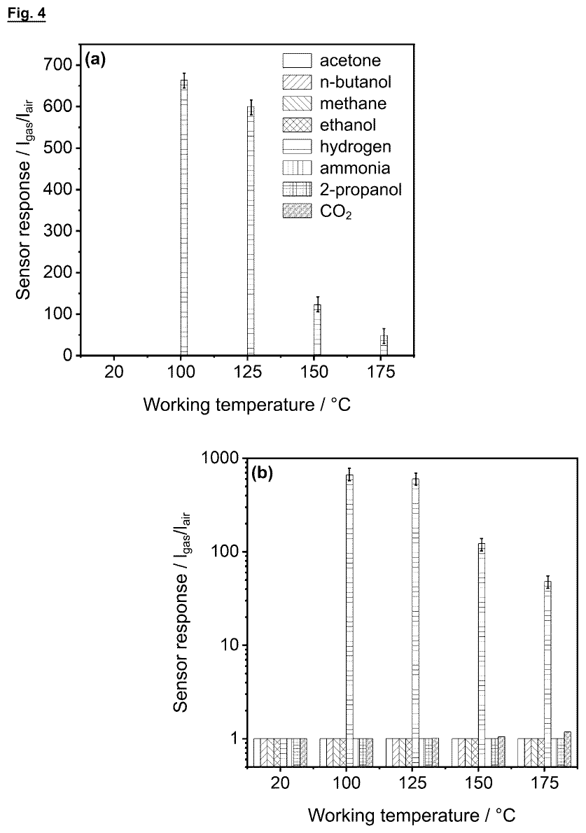

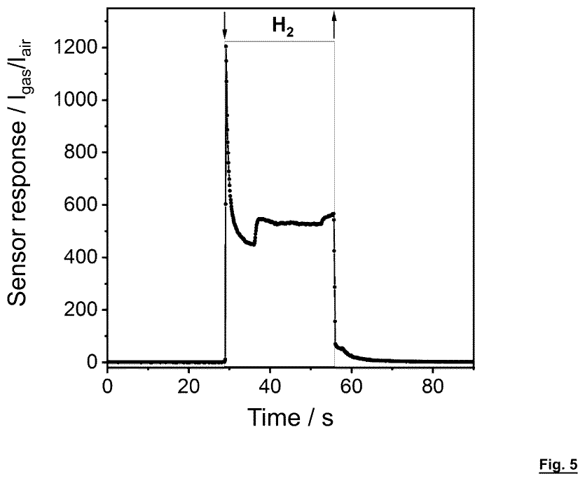

The response behavior of the sensor according to the invention was tested for the gases hydrogen, methane, acetone, ethanol, 2-propanol, n-butanol, ammonia and CO₂ tested in the temperature range 20175° C. The particularly high selectivity towards hydrogen is shown in FIG. 4. It can be seen that none of the other analytes tested reacts with the sensor (S=1). Therefore, the selectivity towards hydrogen is absolute. High sensitivity (=high sensor response) is already evident at 100° C. The selectivity is maintained even at higher temperatures, up to 175° C.

FIG. 5 shows the short response times and regeneration times in the range of seconds of the sensor according to the invention.

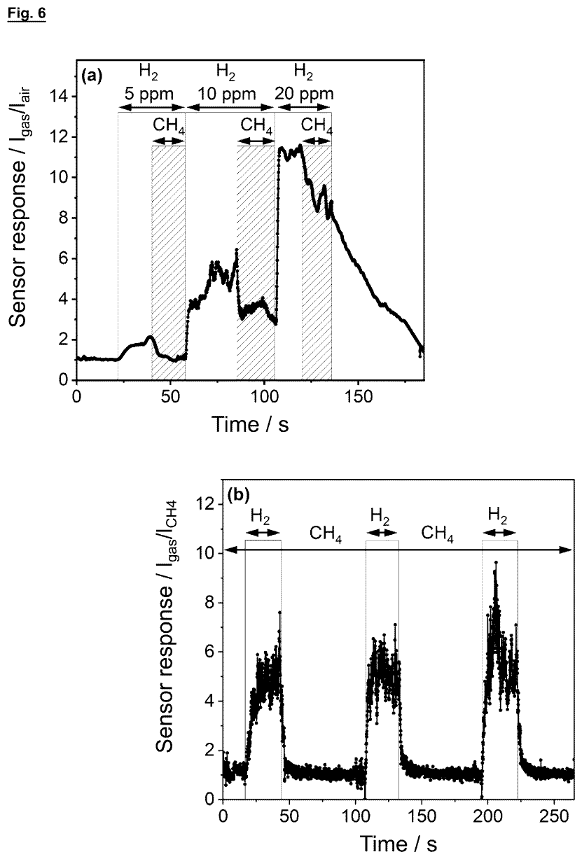

The sensor according to the invention shows sensitivity and selectivity towards H₂ even in an oxygen-free atmosphere. In this case, measurements were performed in a CH4 atmosphere. The results are shown in FIG. 6. This result is very surprising, since, with the prior art semiconductor sensors with the sensor material ZnO@ZIF-8, oxygen must be present in the atmosphere for the measurement of H₂. It turns out that although the sensor response is reduced compared to the air atmosphere, it is still high enough for reliable detection in methane atmosphere.

Zinc oxide structures coated with ZIF-8, which are intended as sensor material for the detection of hydrogen, are known from the following publications, which are incorporated herein by reference.
1) M. Drobek, J.-H. Kim, M. Bechelany, C. Vallicari, A. Julbe, S. S. Kim, MOF-Based Membrane Encapsulated ZnO Nanowires for Enhanced Gas Sensor Selectivity, ACS Appl. Mater. Interfaces. 8 (2016) 8323-8328. https://doi.org/10.1021/acsami.5b12062
2) P. Ji, X. Hu, R. Tian, H. Zheng, J. Sun, W. Zhang, J. Peng, Atom-economical synthesis of ZnO@ZIF-8 core-shell heterostructure by dry gel conversion (DGC) method for enhanced H2 sensing selectivity, J. Mater. Chem. C. 8 (2020) 2927-2936. https://doi.org/10.1039/C9TC06530J
3) X. Wu, S. Xiong, Z. Mao, S. Hu, X. Long, A Designed ZnO@ZIF-8 Core-Shell Nanorod Film as a Gas Sensor with Excellent Selectivity for H2 over CO, Chem.—Eur. J. 23 (2017) 7969-7975. https://doi.org/10.1002/chem.201700320
4) F. Cui, W. Chen, L. Jin, H. Zhang, Z. Jiang, Z. Song, Fabrication of ZIF-8 encapsulated ZnO microrods with enhanced sensing properties for H2 detection, J. Mater. Sci. Mater. Electron. 29 (2018) 19697-19709. https://doi.org/10.1007/s10854-018-0095-9
5) R. Lv, Q. Zhang, W. Wang, Y. Lin, S. Zhang, ZnO@ZIF-8 Core-Shell Structure Gas Sensors with Excellent Selectivity to H2, Sensors. 21 (2021) 4069. https://doi.org/10.3390/s21124069
6) A. I. Khudiar, A. K. Elttayef, M. K. Khalaf, A. M. Oufi, Fabrication of ZnO@ZIF-8 gas sensors for selective gas detection, Mater. Res. Express. 6 (2020) 126450. https://doi.org/10.1088/2053-1591/ab69c2

The following Table 6 provides an overview of the respective sensor material in comparison to the inventive material and to ZnO without conversion layer.

TABLE 6
Overview of known ZnO@ZIF-8 sensor materials and
ZnO without conversion layer (ZnO) as sensor material
compared to the sensor material according to the invention

| | | | | Temperature during | |
|---|---|---|---|---|---|
| | | Maximum sensor | | measurement Working | |
| | | response (Rₐᵢᵣ/R_{gas} = | | temperature of the | |
| Publication | Gas | I_{gas}/Iₐᵢᵣ) | | sensor | |
| Drobek et | H₂ | 2.34 | (10 ppm) (ZnO) | 300° | C. |
| al.[1] | | 1.17 | (10 ppm) | | |
| | C₇H₈ | 1.24 | (10 ppm) | | |
| | C₆H₆ | 1.33 | (10 ppm) | | |
| Ji et al.[2] | H₂ | 2.4 | (100 ppm) | 241.7° | C. (5 V) |
| | | 2.3 | (100 ppm) (ZnO) | | |
| | C₇H₈ | 2.6 | (10 ppm) | | |
| | | 4.1 | (10 ppm) (ZnO) | | |
| | EtOH | ~4.5 | (100 ppm) | | |
| | | 13.5 (100 ppm) | | | |
| | | (ZnO) | | | |
| | AcOH | ~4.0 (100 ppm) | | | |
| | | ~8.0 (100 ppm) | | | |
| | | (ZnO) | | | |
| | CO₂ | ~1.0 | (100 ppm) | | |
| | | 2.9 | (100 ppm) (ZnO) | | |
| | Formaldehyde | ~5.5 (100 ppm) | | | |
| | | ~9.5 (100 ppm) | | | |
| | | (ZnO) | | | |
| | Xylene | 0.5 | (100 ppm) | | |
| | | 2.0 | (100 ppm) (ZnO) | | |
| Wu et al.[3] | H₂ | 3.28 | (50 ppm) | 250° | C. |
| | | 2.15 | (50 ppm) (ZnO) | | |
| | CO | ~1.1 | (50 ppm) | | |
| | | ~1.6 | (50 ppm) (ZnO) | | |
| Cui et al.[4] | H₂ | ~4.0 | (50 ppm) (ZnO) | 125° | C. |
| | H₂ | ~8.6 | (50 ppm) | | |
| | | ~9.5 | (100 ppm) | | |
| | CO | ~2.4 | (50 ppm) | | |
| | CH₄ | ~2.2 | (50 ppm) | | |
| | C₂H₆ | ~1.4 | (50 ppm) | | |
| | C₂H₄ | ~2.2 | (50 ppm) | | |
| | C₂H₂ | ~4.0 | (50 ppm) | | |
| Lv et al.[5] | H₂ | ~1 | (100 ppm) (ZnO) | 150-190° | C. |
| | H₂ | ~3.7 (100 ppm) | | 290° | C. |
| | | (ZnO) | | | |
| | H₂ | ~1.1 | (100 ppm) | 150-190° | C. |
| | H₂ | ~5.8 | (100 ppm) | 290° | C. |
| | C₂H₆O | ~1.25 (100 ppm) | | 150° | C. |
| | | (ZnO) | | | |
| | C₂H₆O | ~2.9 (100 ppm) | | 290° | C. |
| | | (ZnO) | | | |
| | C₃H₆O | ~1.3 | (50 ppm) (ZnO) | 150° | C. |
| | C₃H₆O | ~3.2 | (50 ppm) (ZnO) | 290° | C. |
| Khudiar et | H₂ | ~6.25 (50 ppm) | | 275° | C. |
| al.[6] | | (converted) | | | |
| | C₆H₆ | ~0 | (50 ppm) | | |
| According to | H₂ | 546 | (100 ppm) | 100° | C. |
| the invention | CH₄ | 1 | (100 ppm) | 100-175° | C. |
| Sensor | Acetone | 1 | (100 ppm) | 100-175° | C. |
| material | n-butanol | 1 | (100 ppm) | 100-175° | C. |
| t-ZnO@ZIF-8 | 2-propanol | 1 | (100 ppm) | 100-175° | C. |
| | Ethanol | 1 | (100 ppm) | 100-175° | C. |
| | NH₃ | 1 | (100 ppm) | 100-175° | C. |
| | CO₂ | 1 | (100 ppm) | 100° | C. |
| | | 1.01 | (100 ppm) | 125° | C. |
| | | 1.05 | (100 ppm) | 150° | C. |
| | | 1.18 | (100 ppm) | 175° | C. |
| | H₂/CH₄ | 3.4 (10 ppm H₂ + | | 150° | C. |
| | | 10 ppm CH₄) | | | |
| | H₂/CH₄ | 8.7 (10 ppm H₂ + | | | |
| | | 20 ppm CH₄) | | | |
| | H₂ Measurement | 5.1 (100 ppm H2) | | 100° | C. |
| | in an oxygen- | | | | |
| | free atmosphere | | | | |
| t-ZnO | H₂ | 25 | (100 ppm) | 100° | C. |

It can be clearly seen that, with the hydrogen sensor material according to the invention, the sensor response at 100 ppm H₂ and 100° C. measurement temperature in the presence of oxygen is more than 100 (546) and is therefore a factor of 50 to 100 higher than that of the reference materials. Furthermore, it can be seen that the selectivity of the sensor material according to the invention is extremely good. There is no sensor response for the other gases in this case methane, acetone, ethanol, 2-propanol, n-butanol, ammonia and CO₂ and therefore no cross-sensitivity, i.e., maximum selectivity.

The measurement of H₂ in an oxygen-free atmosphere is also possible.

### Materials and Methods

Information on the chemicals and equipment used is summarized in Tables 7 and 8.

TABLE 7
List of reagents used in the synthesis, including manufacturer.

| Reactant | Purity | Manufacturer |
|---|---|---|
| 2-methylimidazole (HMelM) | 99% | Sigma-Aldrich |

TABLE 8
Measurement methods and devices used

| | |
|---|---|
| X-ray powder | PXRD measurements of the synthesized samples: Empyrean |
| diffractometer PXRD | MPD diffractometer (PAN′alytical) with PIXcel detector, Cu-K_{α1, 2} |
| | radiation and Debye-Scherrer geometry |
| | PXRD comparison measurements after sorption and TG |
| | measurements: Stoe Stadi MP diffractometer with Mythen 1K |
| | detector and Cu-K_{α1} radiation in transmission geometry |
| Thermogravimetric (TG) | Linseis STA 1600 with a heating rate of 4 K/min in air flow (100 |
| measurements | sccm/min). |
| N2-sorption | BELSorp Max from BEL Japan. Before the measurement, the |
| measurements | samples were heated for 16-24 h at 100° C. and under reduced |
| | pressure (<10⁻² kPa). |
| Scanning electron | FSEM Supra55VP (Zeiss, Germany) |
| microscope (SEM) | |

Preparation of t-ZnO@ZIF-8

For the synthesis, approximately 200 mg of ZnO tetrapods (2.46 mmol) are placed into one half of a 30 mL Teflon reactor. 200-400 mg of 2-methylimidazole (HMeIM) (2.44-4.87 mmol) are placed in the reactor in a smaller Teflon vessel. The reactor is sealed in a steel autoclave. The reaction is carried out for 2-60 hours at 140° C. After the reaction, the reactor is quickly cooled and opened. The product is processed at 100° C. and under reduced pressure (<10⁻² kPa) to remove excess HMeIM from the samples. The exact reaction conditions are summarized in Table 9 and the general synthesis structure is shown in FIG. 2.

TABLE 9
Synthesis details for the solvent-free
coating of ZnO tetrapods with ZIF-8.

| | | |
|---|---|---|
| Starting material | Reaction conditions | Post-treatment |

| t-ZnO | HMelM | T | T | t | T |
|---|---|---|---|---|---|
| mg | mg | H | ° C. | h | ° C. |
| 200 | 200 | 2 | 140 | 6 | 100 |
| | | 3 | | 6 | |
| | | 4 | | 8 | |
| | | 5 | | 8 | |
| | | 6 | | 8 | |
| | | 7 | | 22 | |
| | | 8 | | 22 | |
| | | 9 | | 22 | |
| | | 10 | | 32 | |
| | 400 | 15 | | 32 | |
| | | 20 | | 32 | |
| | | 30 | | 44 | |
| | | 40 | | 30 | |
| | | 50 | | 30 | |
| | | 60 | | 30 | |

### Analytics

The homogeneity of the coating was checked using SEM images (FIG. 1) and the layer thickness of the ZIF-8 layers was determined from four samples and is summarized in Table 1. The quantitative determination of the ZIF-8 content was carried out by thermogravimetric analysis. The samples contain the amounts of ZIF-8 listed in Table 2. The specific surface areas were determined by nitrogen sorption measurement and are listed in Table 3. For the measurement of hydrogen detection of the sample t-ZnO@ZIF-8(4h), an arm of a single tetrapod was connected by means of the focused ion beam (FIB) method using platinum on a SiO₂/silicon wafer to gold contacts to measure the current flow in a single arm when exposed to different sample gases. The response behavior of the sensor was tested for the gases hydrogen, methane, acetone, ethanol, 2-propanol, n-butanol, ammonia and CO₂ tested in the temperature range 20-175° C. The particularly high selectivity towards hydrogen is shown in FIG. 4. High sensitivity (=high gas response) is already evident at 100° C. The selectivity is maintained even at higher temperatures, up to 175° C. Likewise, short response times and regeneration times (FIG. 5) in the range of seconds are observed. The observed sensitivity and selectivity towards H₂ is atypical for semiconductor sensors in a CH₄ atmosphere (FIG. 6).

### Sensor Manufacturing and Testing

The t-ZnO@ZIF-8 tetrapods or single microrods were contacted with platinum via focused ion beam (FIB) on a SiO2/silicon wafer with gold contacts using a process developed by Lupan et al. [O. Lupan, V. Cretu, M. Deng, D. Gedamu, I. Paulowicz, S. Kaps, Y. K. Mishra, O. Polonskyi, C. Zamponi, L. Kienle, V. Trofim, I. Tiginyanu, R. Adelung, Versatile Growth of Freestanding Orthorhombic α-Molybdenum Trioxide Nano- and Microstructures by Rapid Thermal Processing for Gas Nanosensors, J. Phys. Chem. C. 118 (2014) 15068-15078. https://doi.org/10.1021/jp5038415]

[O. Lupan, L. Chow, Th. Pauporte, L. K. Ono, B. Roldan Cuenya, G. Chai, Highly sensitive and selective hydrogen single-nanowire nanosensor, Sens. Actuators B Chem. 173 (2012) 772-780. https://doi.org/10.1016/j.snb.2012.07.111.]

They were tested in air with a range of gases: acetone, n-butanol, methane, ethanol, hydrogen, ammonia, 2-propanol and CO₂ at various operating temperatures from 20 to 175° C. using 100 ppm of the previously reported analytes.

The operating temperature was limited to a maximum temperature of 175° C., which is well below the decomposition temperature of the MOF.

The sensor response(S) was calculated using the ratio of the current with gas exposure (I-gas) and in air (I-air): S=I_{gas/I-air}. For sensor response experiments in air with H₂/CH₄ mixtures, the H₂ concentration was gradually increased from 5 to 10 to 20 ppm. After equilibration in each step, 10 ppm CH₄ were added for a short time. In addition, a sensor response experiment of H₂ in pure CH₄ as carrier gas with 100 ppm H₂ was carried out. For these measurements, only samples formed after 4 h response time (−t-ZnO@ZIF8(4h)) were used.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

1. A hydrogen sensor material comprising:
at least one rod-shaped single crystal with a wurtzite structure made of a semiconductor and a conversion layer formed by a MOF on the surface of the single crystal,
wherein the single crystal has a length of 100 nm to 100 μm and a lateral extent of 500 nm to 10 μm, and
wherein an aspect ratio of the single crystal of length to lateral extent is greater than 3.

2. The hydrogen sensor material according to claim 1, wherein the rod-shaped single crystal with wurtzite structure is formed from zinc oxide (ZnO) or ZnTe or ZnSe.

3. Hydrogen sensor material according to claim 1, characterized in that the conversion layer of the MOF ZIF-8 ([Zn(C₄H₅N₂)₂)]) is formed.

4. The hydrogen sensor material according to claim 1, wherein the rod-shaped single crystal with wurtzite structure is formed from tetrapodal zinc oxide (t-ZnO).

5. The hydrogen sensor material according to claim 1, wherein the sensor response at 100 ppm H₂ and 100° C. measurement temperature in the presence of oxygen is more than 100.

6. The hydrogen sensor material according to claim 1, wherein the sensor reaction to the gases methane, acetone, ethanol, 2-propanol, n-butanol, ammonia and CO₂ at 100 ppm and 100° C. measurement temperature in the presence of oxygen is 1.

7. A hydrogen sensor material structure comprising the hydrogen material according to claim 1, wherein at least two rod-shaped single crystals with wurtzite structure are connected to form a diffusion-open network.

8. A method for producing the hydrogen sensor material and/or structure according to any claim 1, the method comprising:
I. determining a reaction temperature in a range between 60° C. and 160° C., the reaction time and a molar ratio in relation to the desired layer thickness using Tables 1 and 2 and the RGT rule:
TABLE 1
ZnO to HMelM ratio

| reaction time/h | ZnO | HMelM |
|---|---|---|
| 4 | 50 | 1 |
| 5 | 40 | 1 |
| 10 | 25 | 1 |
| 15 | 25 | 2 |
| 20 | 10 | 1 |
| 30 | 6 | 1 |
| 40 | 5 | 1 |
| 50 | 10 | 3 |
| 60 | 2 | 1 |

TABLE 2
Average film thickness of the ZIF-8 coating that can be achieved
at a reaction temperature of 140° C. for a given reaction time.

| | Average layer thickness at a reaction | |
|---|---|---|
| reaction time/h | temperature of 140° C./nm | |
| 4 | 60 | (±20) |
| 8 | 160 | (±80) |
| 10 | 210 | (±110) |
| 15 | 320 | (±160) |
| 20 | 430 | (±220) |

I. placing ZnO material into a sealable reactor;
III. adding 2-methylimidazole (HMeIM);
IV. closing the reactor;
V. controlling a temperature of the reactor; and
VI. cooling the reactor,
wherein steps II and Ill are adapted to be carried out in a variable order.

9. The method for producing a hydrogen sensor material according to claim 8, wherein excess HMeIM is removed by treating the crude product under reduced pressure and/or at elevated temperature.

10. The hydrogen sensor material according to claim 1, wherein the hydrogen sensor material is used at a measurement temperature of less than 120° C.

11. The hydrogen sensor material according to claim 1, wherein the hydrogen sensor material is in an oxygen-free atmosphere.
